(19) United States
(12) Patent Application Publication
(10) Pub. No.: US 2015/0047451 A1
(43) Pub. Date: Feb. 19, 2015

(54) ARTICULATION FOR SURGICAL EQUIPMENT USING BALL JOINT

(71) Applicants: INDUSTRY-UNIVERSITY COOPERATION FOUNDATIONN HANYA- NG UNIVERSITY ERICA CAMPUS, Ansan-si Gyeonggi-do (KR); KOHYOUNG TECHNOLOGY INC., Seoul (KR)

(72) Inventor: YoungSik Kwon, Ansan-si (KR)

(73) Assignees: INDUSTRY-UNIVERSITY COOPERATION FOUNDATION HANYANG UNIVERSITY ERICA CAMPUS, Ansan-si Gyeonggi-do (KR); KOHYOUNG TECHNOLOGY INC., Seoul (KR)

(21) Appl. No.: 14/005,100

(22) PCT Filed: Apr. 17, 2013
(86) PCT No.: PCT/KR2013/003252

§ 371 Date: Sep. 13, 2013

(30) Foreign Application Priority Data
Apr. 27, 2012 (KR) 10-2012-0044938

(51) Int. Cl. A61B 17/00; B25J 17/00

(52) U.S. Cl. CPC A61B 17/00; B25J 17/00; A61B 2017/00314; A61B 2017/00477
USPC 74/490.05

(57) ABSTRACT

Provided is an articulation for surgical equipment using a ball joint fastened by the ball joint. The present invention has been made in an effort to provide an articulation for surgical equipment using a ball joint of which the shaft of pitching or yawing is not changed even though the pitching or the yawing is repeated. The articulation for surgical equipment using a ball joint is configured to include a plurality of disks; a ball joint to allow the plurality of disks to be pitched or yawed between the plurality of disks based on a longitudinal direction of the articulation for surgical equipment; and a driving wire applying a driving force to pitch, yaw, or roll the plurality of disks. According to the articulation for a surgical equipment using a ball joint according to the embodiment of the present invention, the rotating shaft of the articulation is not changed despite the repeated movement of the articulation, and since the durability of the articulation is increased, the safety of the surgical equipment can be improved when the articulation is applied to the surgical equipment.

### TECHNICAL FIELD

The present invention relates to an articulation for surgical equipment using a ball joint, and more particularly, to an articulation for surgical equipment using a ball joint, in which the articulations for the surgical equipment are fastened by the ball joint.

### BACKGROUND ART

In recent, with the development of minimally invasive surgery, a quantity of tissues of a human body damaged at the time of diagnosis or surgical treatment decreases such that effects, such as reduction in a recovery time of a patient and reduction in a side effect can be obtained.

An example of the general minimally invasive surgery type may include an endoscopy using an endoscope and an example of a surgery using the endoscopy may representatively include a laparoscopic surgery using a laparoscope. A general laparoscopic surgery is performed within the abdomen by expanding a patient's abdomen with gas, slightly cutting a patient's skin, inserting a laparoscopic surgical equipment into a patient's abdomen, and controlling an end-effector at a tip of the laparoscopic surgical equipment.

However, to perform the laparoscopic surgery, there is a need to insert the laparoscopic surgical equipment into a patient's abdomen to allow the end-effector to access affected areas. In this case, when the laparoscopic surgical equipment has a hard bar type, it may cause a damage to internal organs of an abdominal cavity. Therefore, the laparoscopic surgical equipment is configured so that the end-effector may perform a tri-axis rotation motion by coupling the end-effector with a distal end of the shaft generally configured of poly-articulation. Herein, the tri-axis rotation motion means pitching to allow the end-effector to perform a bending motion in a vertical direction at a tip of the laparoscopic surgical equipment, yawing to allow the end-effector to perform a bending motion in a horizontal direction, and rolling to allow the end-effector to perform a rotation motion in a longitudinal direction of a shaft, in which the pitching and the yawing may be performed by bending the articulation of the shaft and the rolling may be performed the overall rotation of the shaft.

In connection with this, U.S. Pat. No. 6,817,974 discloses a wrist of the surgical equipment to allow the end-effector to pitched or yawed. The wrist of the surgical equipment is configured of a plurality of disks, both sides of the disk are provided with hinges, and the disks are coupled with each other by intersecting the hinges so as to perform the pitching or the yawing. However, as described above, when the disks are coupled with each other by the hinges, hinge pins are worn due to the movement of the hinge by the motion of the disk and thus the hinge shaft moves or is deformed to deviate the pitching or the yawing of the surgical equipment from an originally set direction, such that the precision control of the surgical equipment may not be performed.

### RELATED ART DOCUMENT

### Patent Document

(Patent Document 1) U.S. Pat. No. 6,817,974 B1 (Feb. 20, 2003)

### DISCLOSURE

### Technical Problem

An object of the present invention is to provide an articulation for surgical equipment using a ball joint of which the shaft of pitching or yawing is not changed even though the pitching or the yawing is repeated.

### Technical Solution

In one general aspect, there is provided an articulation for surgical equipment using a ball joint, including: a plurality of disks; a ball joint to allow the plurality of disks to be pitched or yawed between the plurality of disks based on a longitudinal direction of the articulation for surgical equipment; and a driving wire applying a driving force to pitch, yaw, or roll the plurality of disks.

The disk may be provided with a plurality of through holes through which driving wires pass to be adjacent to a circumference thereof and a center of the disk may be provided with a hollow through which a power line or a control line required to drive and control an end-effector passes.

### Advantageous Effects

As set forth above, According to the articulation for surgical equipment using a ball joint according to the embodiments of the present invention, the rotating shaft of the articulation is not changed despite the repeated movement of the articulation, and since the durability of the articulation is increased, the safety of the surgical equipment can be improved when the articulation is applied to the surgical equipment.

### DESCRIPTION OF DRAWINGS

The above and other objects, features and advantages of the present invention will become apparent from the following description of preferred embodiments given in conjunction with the accompanying drawings, in which:

FIG. 1 is a diagram illustrating an articulation for surgical equipment using a ball joint according to a first embodiment of the present invention;

FIG. 2 is a perspective view of two types of disk;

FIG. 3 is a diagram illustrating an example of the articulation for surgical equipment using a ball joint according to the first embodiment of the present invention;

FIG. 4 is a side view of the articulation for surgical equipment according to the first embodiment of the present invention;

FIG. 5 is a cross-sectional view of an articulation for surgical equipment according to a second embodiment of the present invention;

FIG. 6 is a perspective view of a link part; and

FIG. 7 is a cross-sectional view of a state in which the articulation for surgical equipment using a ball joint illustrated in FIG. 5 is bent.

### BEST MODE

Hereinafter, an articulation for surgical equipment using a ball joint according to embodiments of the present invention will be described in more detail with reference to the accompanying drawings.

### First Embodiment

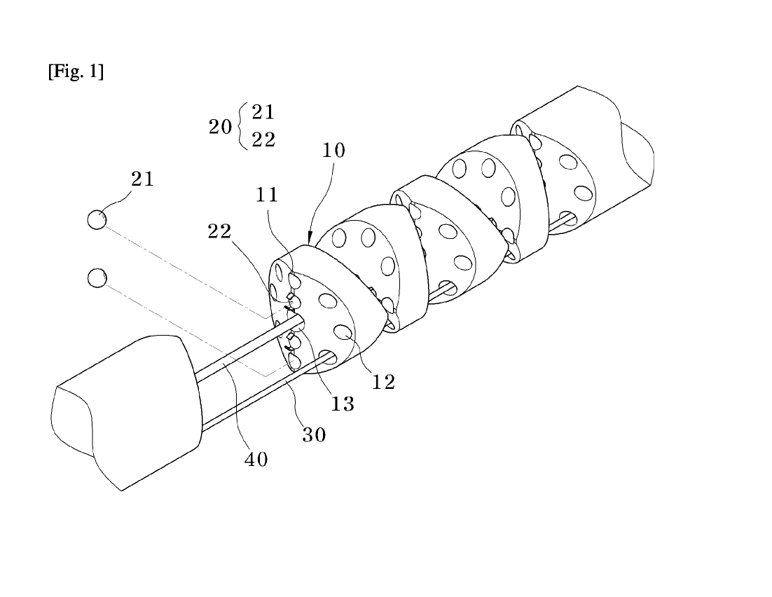

FIG. 1 is a diagram illustrating an articulation for surgical equipment according to a first embodiment of the present invention.

The articulation for surgical equipment according to the embodiment of the present invention may be configured to include a plurality of disks 10, ball joints 20 to allow the plurality of disks 10 to be pitched or yawed between the plurality of disks 10 based on a longitudinal direction of the articulation for surgical equipment, and a driving wire 30 generating a driving force to allow the plurality of disks 10 to pitched, yawed, or rolled (hereinafter, the longitudinal direction of the articulation for surgical equipment is set to be a reference direction, which is described as ‘pitching’, ‘yawing’, and ‘rolling’).

One of the diameters of circles for the both surfaces of the disk, respectively, further protrudes than a circumference thereof to easily connect between the disks 10 by the ball joint 20, thereby forming a protruding part 11 having a shape in which the diameter of the protruding circle is similar to a mountain ridge. The disk 10 may be divided into two types according to the disposition of the protruding parts 11 on both surfaces of the disk and the articulation for surgical equipment using the ball joint according to the embodiment of the present invention may be configured by combining the two types of disks.

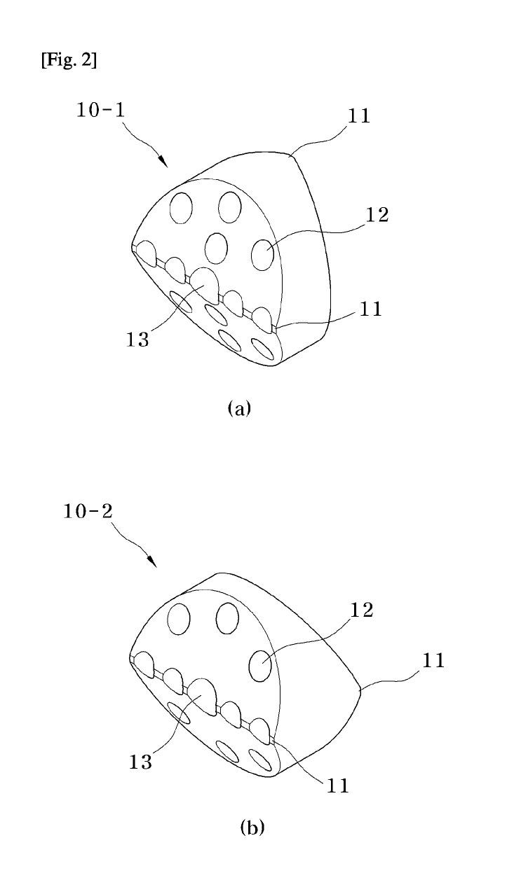

FIG. 2 is a perspective view of the two types of disk 10. FIG. 2A illustrates a disk (hereinafter, referred to as ‘first type of disk 10-1’) in which an angle between a ridge of the protruding part 11 on one surface of the disk and a ridge of the protruding part 11 on an opposite surface thereof forms 90° and FIG. 2B illustrates a disk (hereinafter, referred to as ‘second type disk 10-2’) in which the ridge of the protruding part 11 on one surface of the disk is parallel with the ridge of the protruding part 11 on an opposite surface thereof. The first type of disk 10-1 and the second type of disk 10-2 may be selectively adopted to satisfy a radius of curvature required in the surgical equipment.

First, the first type of disk and the second type of disk will be described. Since the rotating shafts of the articulations on the first type of disk 10-1 form an angle of 90°, for example, one side thereof performs the pitching and the other side thereof performs the yawing by setting the first type of disk 10-1 as a boundary. On the other hand, since the rotating shafts of the articulations on the second type of disks 10-2 are parallel with each other, the pitching and the yawing are not switched between one surface and the other surface thereof by setting the second type of disk 10-2 as the boundary and the pitching or the yawing is maintained. Therefore, when the articulation for surgical equipment is formed using the second type of disk 10-2, the radius of curvature of the articulation becomes small. Therefore, the second type of disk 10-2 may be specially used for the surgical equipment that performs an operation in the narrow space.

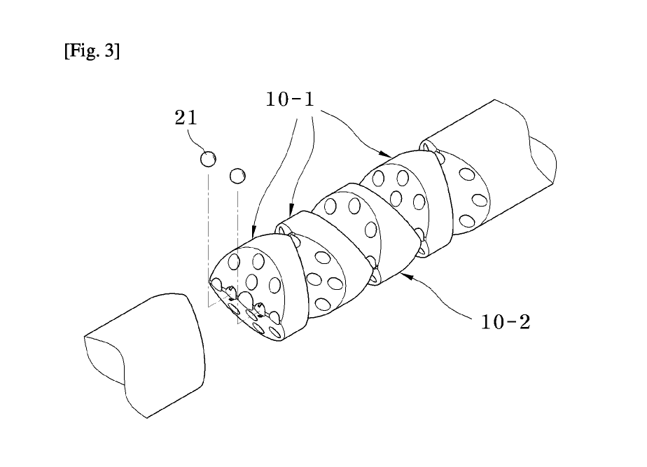

FIG. 3 is a diagram illustrating an example of the articulation for surgical equipment using a ball joint according to the first embodiment of the present invention and illustrates the articulation for surgical equipment in which the first type of disk 10-1—the first type of disk 10-1—the second type of disk 10-2—the first type of disk 10-1 are sequentially coupled with each other by the ball joint.

Referring back to FIG. 1, a plurality of through holes 12 through which a driving wires 30 transferring driving force to allow the disk to be pitched, yawed, and rolled passes are disposed to be adjacent to the circumference of the disk 10 and a center of the disk is provided with a hollow 13 through which a power line or control line 40 required to drive and control the end-effector may pass. Herein, at least three through holes 12 need to be formed. The reason is that when the driving wire 30 is one or two, the disk 10 may not be pitched or yawed in a desired direction. Therefore, for the precision control of the surgical equipment, each disk 10 needs to be provided with at least three through holes 12 and the number of through holes 12 formed on the disk 10 may be appropriately selected in consideration of a cross sectional area of the disk, a cross sectional area of the through hole, a cross sectional area of the hollow, easiness of the control, and the like.

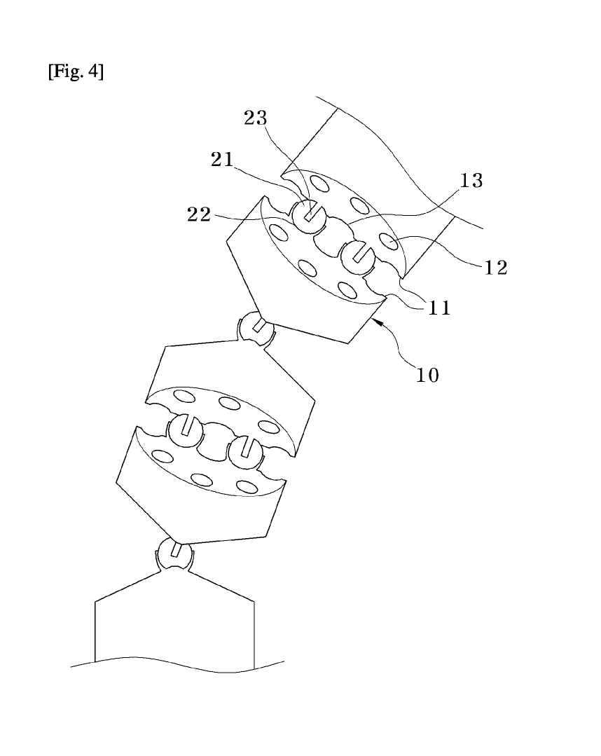

FIG. 4 is a side view of the articulation for surgical equipment according to the first embodiment of the present invention. A ridge of an outside protruding part 11 of the hollow 13 of the disk is provided with two ball receiving grooves 22. The ball receiving groove 22 is provided with a holder 23 enclosing more than half the circumference of the ball so as not to separate that the ball 21 from the ball receiving groove 22. The ball receiving groove 22 on any one surface of the disk 10 has the ball 21 received therein and is enclosed with the holder 23, and the holder 23 of the ball receiving groove 22 on any one surface of another disk 10 encloses the received ball 21 to form the ball join 20 between the disks 10.

The driving wire 30 passes through the through hole 12 formed on the disk 10 to provide the driving force to allow the disk 10 to be pitched, yawed, and rolled and at the same time, serves to apply force so as not to separate the disks 10 coupled by the ball joint 20 from each other. This may be achieved by allowing the driving wire 30 to pull the end-effector at an end of the surgical equipment so as to apply a tensile force from the driving wire 30 to the end-effector, the disk, and the ball joint.

The driving wire 30 applies a tensile force to the disk connected to a distal end of the driving wire in a specific direction to apply a driving force to perform pitching or yawing to the plurality of disk 10 and when the driving wire 30 formed on the disk 10 generally rotates, the plurality of disks 10 rotate accordingly, thereby transferring the driving force to roll the articulation for surgical equipment.

The configuration on how the driving wire 30 is controlled at the outside deviates from the technical scope of the articulation for surgical equipment using a ball joint according to the embodiment of the present invention, and therefore the detailed description thereof will be omitted.

### Second Embodiment

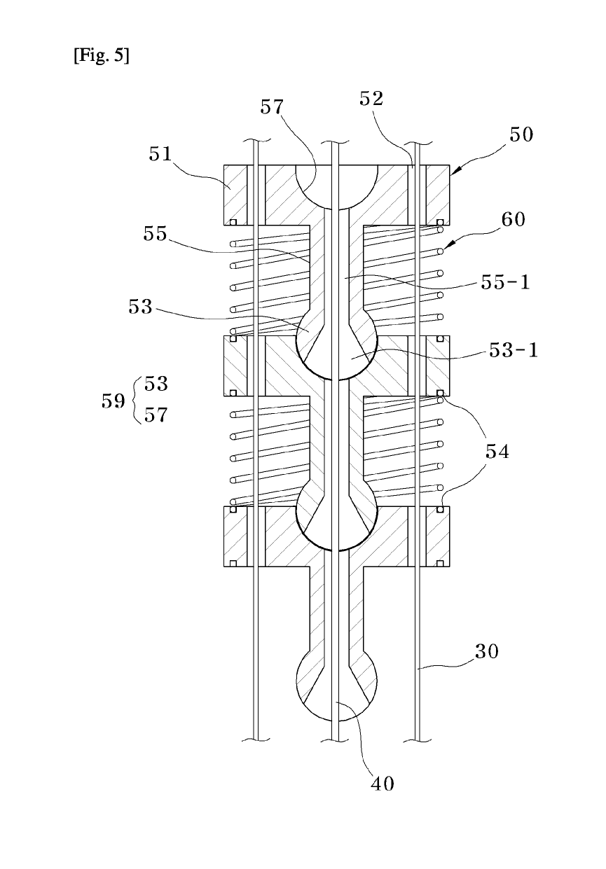

FIG. 5 is a cross-sectional view of an articulation for surgical equipment according to a second embodiment of the present invention.

The articulation for surgical equipment using a ball joint according to another embodiment of the present invention is configured to include a plurality of disks 51, a ball joint 59 to allow the plurality of disks 51 to be pitched or yawed between the plurality of disks, and the driving wire 30 applying the driving force to allow the plurality of disks 51 to be pitched, yawed, or rolled.

According to the articulation for surgical equipment using a ball joint according to the second embodiment of the present invention, a ball 53 is integrally formed with the disk 51 to facilitate the manufacturing of the articulation for surgical equipment. The integrally formed disk 51 and ball 53 are referred to as the link part 50 and the link part 50 may include a pole 55 between the disk 51 and the ball 53 to adjust a distance between the disk 51 and the ball 53. Further, an elastic member 60 applying force to allow the disks 51 to be pulled from each other is provided between the plurality of disks 51, a through hole 52 through which the driving wire 30 passes and a fixed part 54 to which the elastic member is fixed is disposed to be adjacent to a circumference of the disk 51, and a center of the disk 51 is provided with a ball receiving part 57 in which a ball of another disk 51 may be received.

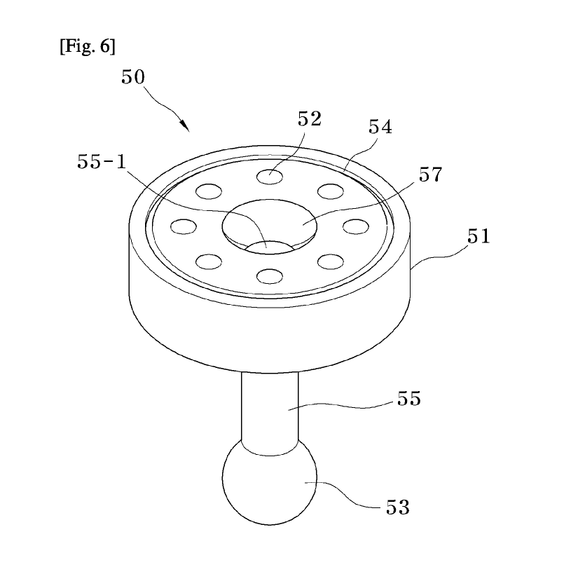

FIG. 6 is a perspective view of the link part 50. The link part 50 is configured to include the disk 51, the ball 53, and a pole 55 connecting the disk and is provided with the through hole 52 and the fixed part 54 to be adjacent to the circumference thereof. Further, a center of an upper surface of the disk 51 is provided with a ball receiving groove 57 receiving a ball 53 of another disk 51 to form a ball joint 59 and the ball 53 and the pole 55 are provided with hollows 53-1 and 55-1 through which a power line or control line 40 required to drive and control the end-effector. In this case, the hollow 53-1 formed in the ball 53 is formed in a cone shape in a circumferential direction from the center of the ball, such that even though the ball 53 rotates within the ball receiving groove 57, it is preferable to prevent the power line or control line 40 from being caught in the ball 53. The pole 55 may be formed to have a length controlled depending on the radius of curvature required for the articulation of surgical equipment using a ball joint.

Referring back to FIG. 5, the ball 53 of the link part is received in the ball receiving groove 57 formed on a disk of another link part to form the ball joint 59. The elastic member 60 is coupled with fixed parts 54 formed on the disk 51 of the link part and the disk 51 of another link part to apply a tensile force so as not to allow the formed ball joint 59 to be separated and at the same time, when the plurality of disks 51 are pitched or yawed by the driving wire 30, the ball joints 59 formed on the disks 51 uniformly rotate, such that the articulation for surgical equipment is generally bent in a smooth curved shape. An example of the elastic member may include a tension spring, but an elastic member to uniformly rotate the ball joints 59 by applying a tensile force so as not to separate the ball joint 59 between the disks 51 may be used, independent of a kind thereof.

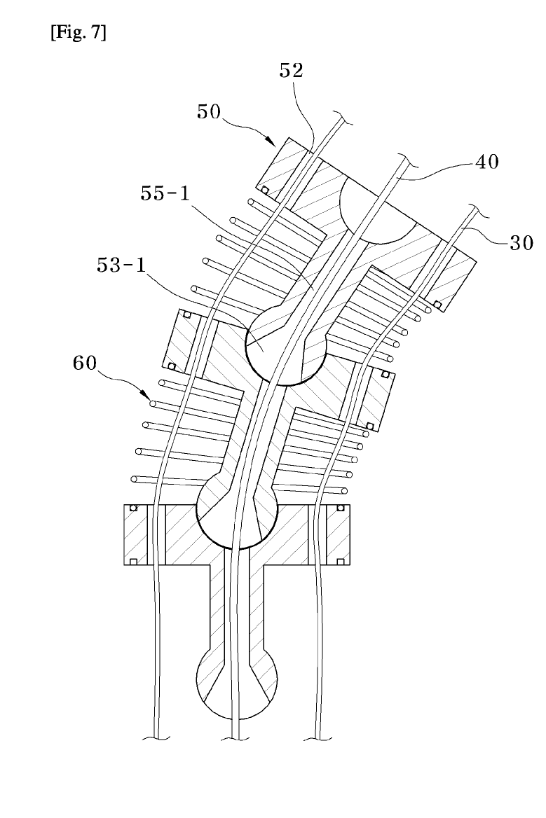

FIG. 7 is a cross-sectional view of a state in which the articulation for surgical equipment using a ball joint illustrated in FIG. 5 is bent. The ball joint uniformly rotates by the elastic member 60, such that the articulation for surgical equipment may be generally bent in a smooth curved form. Further, the power line or control line 40 required to driving and control the end-effector passes through the hollow 53-1 of the ball and the hollow 55-1 of the pole to be connected to the end-effect from the outside without being caught.

### DETAILED DESCRIPTION OF MAIN ELEMENTS

10,51 DISK
10-1 FIRST TYPE OF DISK
10-2 SECOND TYPE OF DISK 11: PROTRUDING PART
12, 52 THROUGH HOLE
13, 53-1, 55-1 HOLLOW
20, 59 BALL JOINT
21, 53 BALL
22, 57 BALL RECEIVING GROOVE 23: HOLDER
30 DRIVING WIRE
40 POWER LINE OR CONTROL LINE REQUIRED TO DRIVE AND CONTROL END-EFFECTOR
50 LINK PART
54 FIXED PART
55 POLE
60 ELASTIC MEMBER

1. An articulation for surgical equipment using a ball joint, comprising:
a plurality of disks;
a ball joint to allow the plurality of disks to be pitched or yawed between the plurality of disks based on a longitudinal direction of the articulation for surgical equipment; and
a driving wire applying a driving force to pitch, yaw, or roll the plurality of disks.

2. The articulation of claim 1, wherein the disk is provided with a protruding part having a shape in which one of the diameters of circles on both surfaces of the disk, respectively, further protrudes than a circumference thereof so that the diameter of the protruding circle has a shape like a mountain ridge.

3. The articulation of claim 2, wherein the plurality of disks are configured of a combination of a first type of disk in which an angle between a ridge of the protruding part on one surface of the disk and a ridge of the protruding part on an opposite surface thereof forms 90° and a second type disk in which the ridge of the protruding part on one surface of the disk is parallel with the ridge of the protruding part on an opposite surface thereof.

4. The articulation of claim 1, wherein the disk is provided with a plurality of through holes through which driving wires pass to be adjacent to a circumference thereof and a center of the disk is provided with a hollow through which a power line or a control line required to drive and control an end-effector passes.

5. The articulation of claim 4, wherein a ridge of an outside protruding part of the hollow is provided with two ball receiving grooves.

6. The articulation of claim 5, wherein the ball receiving groove is provided with a holder enclosing more than half the circumference of the ball so as not to separate the ball therefrom.

7. The articulation of claim 4, wherein at least three through holes are formed.

8. The articulation of claim 1, wherein the disk is integrally formed with a ball received in a ball receiving part of another disk to form the ball joint and a pole disposed between the ball and the disk to control a distance between the ball and the disk.

9. The articulation of claim 8, wherein an elastic member to allow the disks to be applied with a pulling force is disposed between the plurality of disks.

10. The articulation of claim 9, wherein the disk is provided with a through hole through which the driving wire passes to be adjacently disposed at the circumference thereof and a fixed part to which the elastic member is fixed and a center of the disk is provided with a ball receiving part in which a ball of another disk is received.

11. The articulation of claim 8, wherein the ball and the pole are provided with hollows.

12. The articulation of claim 11, wherein the hollow formed in the ball is formed in a cone shape in a circumferential direction from a center of the ball.
